(19) United States
(12) Patent Application Publication
(10) Pub. No.: US 2011/0257605 A1
(43) Pub. Date: Oct. 20, 2011

(54) SELF-CENTERING SYRINGE ASSEMBLY AND CENTERING SYRINGE PLUNGER GUIDE

(75) Inventor: Brian SWEENEY, Las Vegas, NV (US)

(73) Assignee: Kloehn, Inc., Las Vegas, NV (US)

(21) Appl. No.: 13/073,052

(22) Filed: Mar. 28, 2011

Related U.S. Application Data
(60) Provisional application No. 61/325,069, filed on Apr. 16, 2010.

(51) Int. Cl. A61M 5/315

(52) U.S. Cl. 604/220

(57) ABSTRACT

A centering syringe plunger guide (140) includes a guide body (141) including a proximal end (142) and a distal end (149), a coupling bore (143) extending partially into the guide body (141) from the proximal end (142), a precision guide bore (144) extending partially into the guide body (141) from the coupling bore (143), with the precision guide bore (144) adapted to fit over a barrel precision diameter of a syringe barrel and hold the centering syringe plunger guide (140) in a predetermined substantially coaxial alignment with the syringe barrel, and a guide plunger bore (145) extending completely from the precision guide bore (144) to the distal end (149) of the guide body (141). The guide plunger bore (145) is adapted to receive a syringe plunger and is configured to substantially center the syringe plunger in a plunger bore of the syringe barrel.

This application claims benefit of, and priority from, U.S. provisional patent application No. 61/325,069, filed on Apr. 16, 2010 and entitled “Self-Centering Syringe Assembly and Centering Syringe Plunger Guide”

### BACKGROUND OF THE INVENTION

1. Field of the Invention

The invention is related to the field of syringes.

2. Description of the Prior Art

A syringe is a device that includes a plunger configured to move in a bore. The plunger is used to draw in a volume of fluid when the plunger is moved to a retracted position. The volume drawn in is dependent on the cross-sectional area of the bore and also on the stroke length. Conversely, the plunger expels fluid when it is moved to an extended position. Precise movement of the plunger enables the syringe to draw in and expel precise volumes of fluid.

A syringe includes only a single port, wherein fluid is drawn in or expelled out through the single port. Consequently, only one side of the syringe plunger does work. Only one side is substantially closed off and moves and/or pressurizes a fluid being acted on.

A syringe may be used to move and/or meter out fluids. In an automated instrument, for example, a syringe may be used to provide dosing volumes, repeatedly drawing in and expelling fluids. A predetermined fluid volume tolerance may be achieved in the syringe through careful design and construction.

Tolerances are important for the precise metering of fluids. Tolerances are even more important when the fluid volumes being drawn in and expelled are small. Wear and damage may be especially problematic in a small, repetitive-use syringe. Even a small amount of wear may impact the close tolerances required to make such a syringe accurate and reliable in operation. Consequently, leakage and loss may have a disproportionately large impact on the reliability of a small, repetitive-use syringe that is metering out tiny fluid volumes.

Repetitive use may lead to wear. In a syringe having small tolerances between a plunger shaft and bore, any non-linear (i.e., side-to-side) movement of the plunger shaft may lead to contact with the side of the bore and may result in wear or other damage, such as premature wear of the plunger head. The wear to the plunger head may result in contamination of the fluid, for example Small syringe components cannot withstand much wear.

### ASPECTS OF THE INVENTION

In some aspects of the invention, a centering syringe plunger guide comprises:
a guide body including a proximal end and a distal end;
a coupling bore extending partially into the guide body from the proximal end;
a precision guide bore extending partially into the guide body from the coupling bore, with the precision guide bore adapted to fit over a barrel precision diameter of a syringe barrel and hold the centering syringe plunger guide in a predetermined substantially coaxial alignment with the syringe barrel; and
a guide plunger bore extending completely from the precision guide bore to the distal end of the guide body, with the guide plunger bore being adapted to receive a syringe plunger and is configured to substantially center the syringe plunger in a plunger bore of the syringe barrel.

Preferably, the centering syringe plunger guide is formed of a material having predetermined wear characteristics.

Preferably, the centering syringe plunger guide is formed of a plastic material.

Preferably, the centering syringe plunger guide is formed of a bearing-grade plastic material.

Preferably, the guide body further includes a guide textured region on an external circumferential surface.

Preferably, the precision guide bore holds the centering syringe plunger guide in a predetermined substantially coaxial alignment with a plunger bore of the syringe barrel.

Preferably, the precision guide bore holds the centering syringe plunger guide in a predetermined substantially coaxial alignment with a plunger bore of the syringe barrel and wherein the guide plunger bore is smaller than the plunger bore.

Preferably, the centering syringe plunger guide includes a corresponding guide coupling feature in the coupling bore.

Preferably, the precision guide bore of the centering syringe plunger guide is adapted to fit over the barrel precision diameter of the syringe barrel with a predetermined clearance.

Preferably, the coupling bore, the precision guide bore, and the guide plunger bore are substantially coaxial.

Preferably, the coupling bore, the precision guide bore, and the guide plunger bore are substantially coaxial and further are substantially coaxial with a guide body axis BB.

In some aspects of the invention, a self-centering syringe assembly including a centering syringe plunger guide comprises:
a syringe barrel including a plunger bore and a barrel precision diameter formed on one end of the syringe barrel;
a coupling sleeve configured to fit over and be affixed to the barrel precision diameter of the syringe barrel; and
a centering syringe plunger guide configured to be affixed to the coupling sleeve, with the centering syringe plunger guide comprising:
   a guide body including a proximal end and a distal end;
   a coupling bore extending partially into the guide body from the proximal end;
   a precision guide bore extending partially into the guide body from the coupling bore, with the precision guide bore adapted to fit over a barrel precision diameter of a syringe barrel and hold the centering syringe plunger guide in a predetermined substantially coaxial alignment with the syringe barrel; and
   a guide plunger bore extending completely from the precision guide bore to the distal end of the guide body, with the guide plunger bore being adapted to receive a syringe plunger and is configured to substantially center the syringe plunger in a plunger bore of the syringe barrel.

Preferably, the centering syringe plunger guide is formed of a material having predetermined wear characteristics.

Preferably, the centering syringe plunger guide is formed of a plastic material.

Preferably, the centering syringe plunger guide is formed of a bearing-grade plastic material.

Preferably, the guide body further includes a guide textured region on an external circumferential surface.

Preferably, the precision guide bore holds the centering syringe plunger guide in a predetermined substantially coaxial alignment with a plunger bore of the syringe barrel.

Preferably, the precision guide bore holds the centering syringe plunger guide in a predetermined substantially coaxial alignment with a plunger bore of the syringe barrel and wherein the guide plunger bore is smaller than the plunger bore.

Preferably, the coupling sleeve includes a sleeve coupling feature and with the centering syringe plunger guide including a corresponding guide coupling feature in the coupling bore.

Preferably, the precision guide bore of the centering syringe plunger guide is configured to fit over the barrel precision diameter of the syringe barrel with a predetermined clearance.

Preferably, the coupling bore, the precision guide bore, and the guide plunger bore are substantially coaxial.

Preferably, the coupling bore, the precision guide bore, and the guide plunger bore are substantially coaxial and further are substantially coaxial with a guide body axis BB.

### BRIEF DESCRIPTION OF THE DRAWINGS

The same reference number represents the same element on all drawings. It should be understood that the drawings are not necessarily to scale.

FIG. 1 shows a self-centering syringe assembly including a centering syringe plunger guide according to the invention.

FIG. 2 comprises a section view AA of the centering plunger guide according to the invention.

FIG. 3 shows the self-centering syringe assembly partially assembled, with a coupling sleeve positioned on a barrel precision diameter of a syringe barrel.

FIG. 4 shows a full section view AA of the self-centering syringe assembly when partially assembled.

FIG. 5 shows a full section view AA of the self-centering syringe assembly when completely assembled.

### DETAILED DESCRIPTION OF THE INVENTION

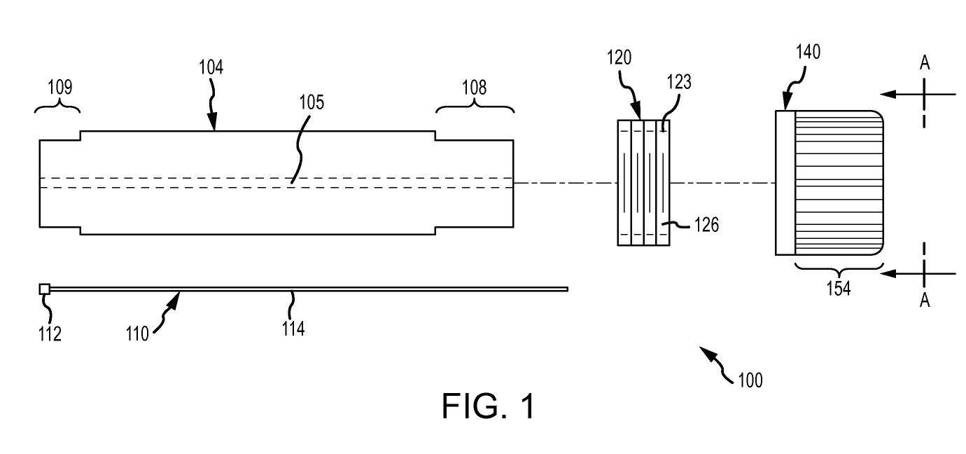

FIGS. 1-5 and the following description depict specific examples to teach those skilled in the art how to make and use the best mode of the invention. For the purpose of teaching inventive principles, some conventional aspects have been simplified or omitted. Those skilled in the art will appreciate variations from these examples that fall within the scope of the invention. Those skilled in the art will appreciate that the features described below can be combined in various ways to form multiple variations of the invention. As a result, the invention is not limited to the specific examples described below, but only by the claims and their equivalents.

FIG. 1 shows a self-centering syringe assembly 100 including a centering syringe plunger guide 140 according to the invention. The self-centering syringe assembly 100 includes a syringe barrel 104, a syringe plunger 110, a coupling sleeve 120, and the centering syringe plunger guide 140.

The syringe barrel 104 includes a plunger bore 105, a barrel precision diameter 108 on one end, and a second barrel precision diameter 109 on the other end. The plunger bore 105 comprises a bore through the syringe barrel 104 that is configured to receive the syringe plunger 110. The plunger bore 105 is preferably substantially cylindrical in shape, but can comprise other cross-sectional shapes. The plunger bore 105 may be substantially coaxial with a central axis of the syringe barrel 104.

The plunger bore 105 is shown as being much smaller in diameter than the outer diameter of the syringe barrel 104, as may be the case where the self-centering syringe assembly 100 is used in some manner of laboratory or industrial instrument that operates on relatively small volumes of fluids. For example, the syringe barrel 104 may be formed of glass and may be employed to meter out small but precise volumes of fluid. In some embodiments, the diameter of the plunger bore 105 may be less than one-eighth inch, for example. In other embodiments, the diameter may be one-quarter inch or less. However, it should be understood that these are just examples, and the plunger bore 105 is not limited to any particular size or size range.

The syringe plunger 110 includes a plunger shaft 114 that is connected to a plunger head 112. The plunger head 112 is configured to sealingly fit into the plunger bore 105 in the syringe barrel 104. The syringe plunger 110 is configured to be reciprocatingly moved in the plunger bore 105.

The plunger shaft 114 in some embodiments is directly and rigidly connected to the plunger head 112. In some embodiments, the plunger shaft 114 and a portion of the plunger head 112 are formed as a single unit. Consequently, any slop in the plunger shaft 114 will result in the plunger head 112 being canted in the plunger bore 105. When the plunger head 112 is misaligned, a sealing contact may be reduced or minimized. Further, misalignment may cause wear of the plunger head 112, the plunger bore 105, and/or the plunger shaft 114. For example, if the syringe barrel 104 is made from glass and the plunger shaft 114 is made from metal or stainless steel, misalignment could cause the plunger shaft 114 to contact the glass, which would cause burnishing or wear of the plunger shaft 114. Such wear to the outer surface of the plunger shaft 114 could subsequently lead to corrosion or oxidation and further degradation and/or contamination. Moreover, wear may result in contamination of the fluid with worn-away particles of the syringe assembly 100.

The coupling sleeve 120 includes a coupling sleeve bore 123 configured to fit over the barrel precision diameter 108. The coupling sleeve 123 may be substantially annular in shape, and may comprise a band that fits over the barrel precision diameter 108. The coupling sleeve 120 further includes a sleeve coupling feature 126 on the exterior.

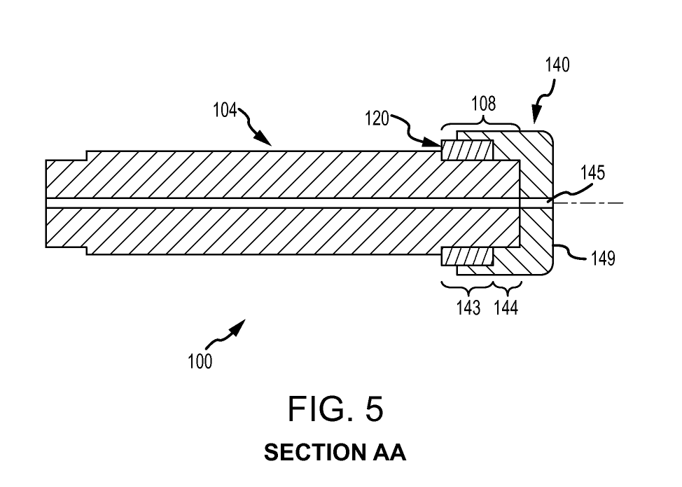

The centering syringe plunger guide 140 fits over the barrel precision diameter 108 and over the coupling sleeve 120 when the coupling sleeve 120 is in place on the barrel precision diameter 108 (see FIG. 5). The centering syringe plunger guide 140 engages the sleeve coupling feature 126 and is retained on the coupling sleeve 120 by the sleeve coupling feature 126.

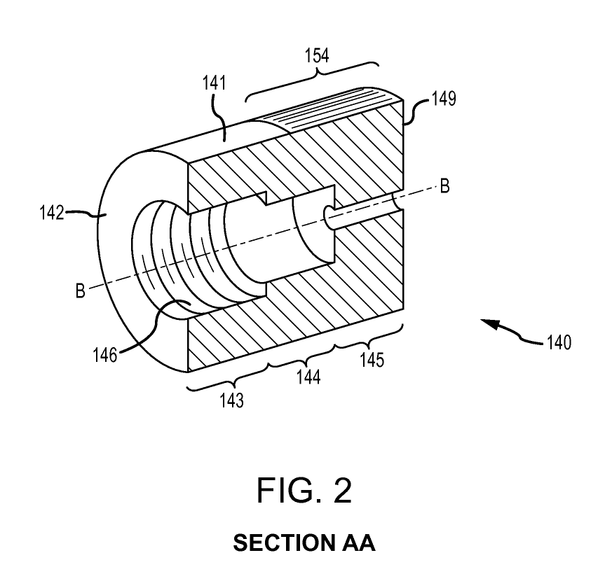

The centering syringe plunger guide 140 includes a guide plunger bore 145 (see FIG. 2) that allows the syringe plunger 110 to pass through the centering syringe plunger guide 140. Consequently, the syringe plunger 110 extends at least somewhat from the syringe barrel 104 and the centering syringe plunger guide 140 when the self-centering syringe assembly 100 is fully assembled.

The centering syringe plunger guide 140 operates to keep the plunger shaft 114 substantially centered in the plunger bore 105. Further, the centering syringe plunger guide 140 retains the syringe plunger 110 in the self-centering syringe assembly 100.

The centering syringe plunger guide 140 may include a guide textured region 154. The guide textured region 154 may provide additional friction and aid a user in grasping and manipulating the centering syringe plunger guide 140. The guide textured region 154 can comprise any manner of surface texturing or roughening, including ridging, grooving, knurling, or irregular surface texturing, for example.

The centering syringe plunger guide 140 is preferably removably affixed to the coupling sleeve 120 by the sleeve coupling feature 126. Alternatively, in some embodiments the centering syringe plunger guide 140 can be permanently affixed to the coupling sleeve 120 by the sleeve coupling feature 126.

The self-centering syringe assembly 100 can comprise a component or sub-assembly of an instrument, such as a laboratory or industrial instrument where fluids are drawn in and expelled by the self-centering syringe assembly 100. The fluids can include any liquids, gasses, or mixtures. To this end, the syringe barrel 104 can include a second barrel precision diameter 109 that can be used to mount or affix the self-centering syringe assembly 100 to another component or assembly, wherein the fluid to be handled is communicated to the syringe barrel 109 at the second barrel precision diameter end.

In an instrument, the self-centering syringe assembly 100 must deliver accurate and reliable operation, even under repeated and long-term use. Wear to the plunger bore 105 may result in leakage at the extreme of plunger retraction travel. Wear to the plunger bore 105 may lead to breakage or damage to the syringe barrel 104. Wear to the plunger head 112 may result in leakage and/or contamination of the fluid. Wear to the plunger shaft 114 may lead to damage to or breakage of the shaft. Wear could further include abrasion to metal components and therefore could permit corrosion or oxidation, which in turn could result in accelerated wear to the plunger head 112 and may further result in leakage and/or contamination of the fluid with corrosion/oxidation by-products.

The self-centering syringe assembly 100 may comprise a physically small component, wherein the plunger bore 105 is of a diameter that requires a thin, rigid plunger shaft 114. For example, the plunger bore 105 in some embodiments may be one-eighth inch or less in diameter. As a result, the plunger shaft 114 may be formed of a metal or other substantially rigid material. Consequently, a plunger shaft 114 made of plastic or other soft materials would not provide a needed rigidity and would likely flex during use, allowing the plunger shaft 114 to contact the sides of the plunger bore 105. Contact with the plunger bore surface may abrade and wear the plunger shaft 114. Contact with the plunger bore surface may abrade and wear the plunger bore 105. Flexing or movement of the plunger shaft 114 may cant the plunger head 112 and cause wear on the plunger head 112 and on corresponding regions of the plunger bore 105. Therefore, it is important that the plunger shaft 114 be kept centered in the plunger bore 105.

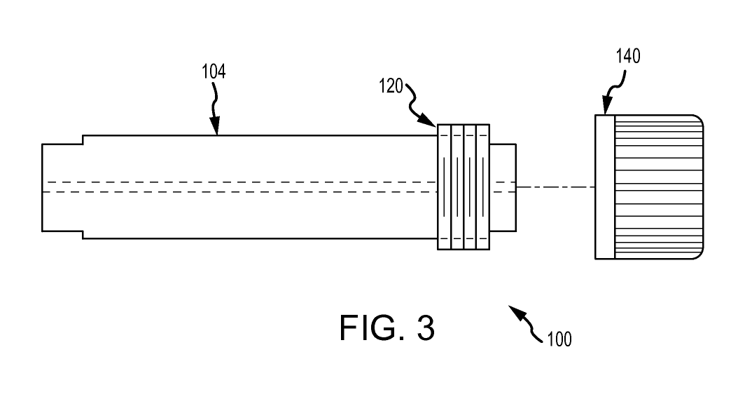

During assembly of the self-centering syringe assembly 100, the syringe plunger 110 is inserted into the plunger bore 105 and the coupling sleeve 120 is placed on the barrel precision diameter 108 (see FIG. 3). The coupling sleeve 120 is configured to be retained on the barrel precision diameter 108. The coupling sleeve 120 may be retained by use of a low tolerance (i.e., frictional) fit. Alternatively, the coupling sleeve 120 may be permanently or removably affixed to the syringe barrel 104, including by glues or bonding agents, welding or other heat processes, shrink fitting, or other suitable process. The centering syringe plunger guide 140 is then affixed to the coupling sleeve 120, wherein the centering syringe plunger guide 140 is retained on the syringe barrel 104 by the coupling sleeve 120. The centering syringe plunger guide 140 includes a guide coupling feature 146 corresponding to the sleeve coupling feature 126, wherein the two coupling features interact to retain the centering syringe plunger guide 140 on the coupling sleeve 120 (see FIG. 2). The centering syringe plunger guide 140 may be removably or permanently affixed to the coupling sleeve 120.

In some embodiments, the guide coupling feature 146 and the sleeve coupling feature 126 comprise threaded surfaces, wherein the centering syringe plunger guide 140 can be rotated and threaded onto and off of the coupling sleeve 120. Alternatively, the guide coupling feature 146 and the sleeve coupling feature 126 can comprise other coupling features, including frictional fit components, resilient or snap fit components, or any other suitable coupling features.

FIG. 2 comprises a section view AA of the centering syringe plunger guide 140 according to the invention. The centering syringe plunger guide 140 comprises a guide body 141 including a proximal end 142 and a distal end 149. The guide body 141 can be substantially cylindrical in shape, as shown, but alternatively could have an exterior in other shapes. For example, the exterior of the guide body 141 could be substantially octagonal in order for the centering syringe plunger guide 140 to be rotated by a wrench or other tool.

The guide body 141 includes three bore sections in the embodiment shown. The bore sections are coaxial along a guide body axis BB. In some embodiments, the guide body axis BB is a substantially central axis of the centering syringe plunger guide 140.

A coupling bore 143 extends partially into the guide body 141 from the proximal end 142. A precision guide bore 144 extends partially through the guide body 141 from the coupling bore 143. A guide plunger bore 145 extends from the precision guide bore 144 to the distal end 149 of the guide body 141. The bores 143, 144, and 145 together create a stepped bore that passes completely through the guide body 141.

In some embodiments, the coupling bore 143, the precision guide bore 144, and the guide plunger bore 145 are substantially coaxial. In some embodiments, the coupling bore 143, the precision guide bore 144, and the guide plunger bore 145 are substantially coaxial and further are substantially coaxial with a guide body axis BB.

The bores 143, 144, and 145 each have a different function. The coupling bore 143 is configured to fit over and interact with the coupling sleeve 120. To this end, the coupling bore 143 includes the guide coupling feature 146.

The precision guide bore 144 is configured to fit over the barrel precision diameter 108 of the syringe barrel 104 and hold the centering syringe plunger guide 140 in a predetermined substantially coaxial alignment with the syringe barrel 104. The precision guide bore 144 holds the centering syringe plunger guide 140 in a predetermined substantially coaxial alignment with the plunger bore 105 of the syringe barrel 104. The precision guide bore 144 of the centering syringe plunger guide 140 is adapted to fit over the barrel precision diameter 108 with a predetermined clearance.

The guide plunger bore 145 is configured to receive the syringe plunger 110. The guide plunger bore 145 in some embodiments may be smaller in diameter than the plunger bore 105, but are still kept in coaxial alignment. The guide plunger bore 145 is configured to substantially center the syringe plunger 110 in the plunger bore 105 of the syringe barrel 104. The guide plunger bore 145 therefore keeps the plunger head 112 properly aligned in the plunger bore 105 and prevents the plunger head 112 from being canted. The guide plunger bore 145 therefore holds the plunger shaft 114 substantially in the center of the plunger bore 105 of the syringe barrel 104. Consequently, the centering syringe plunger guide 140 will center the plunger shaft 114 in the plunger bore 105 even where the plunger shaft 114 may tend to slop up and down during reciprocating motion. The centering syringe plunger guide 140 will center the plunger shaft 114 when the reciprocating motion is provided from a non-linear source, such as from an eccentric wheel, cam or other such device.

Depending on the material and construction of the centering syringe plunger guide 140, the guide plunger bore 145 may comprise a bearing surface that allows the plunger shaft 114 to move easily into and out of the self-centering syringe assembly 100 in a reciprocating motion. The centering syringe plunger guide 140 may be formed of a material having predetermined wear characteristics. For example, the material may better resist or withstand wear than the syringe barrel material. In one example embodiment, the syringe barrel 104 is formed of the hardest material, the plunger shaft 114 is formed of a second hardest material, and the plunger guide 140 and the plunger head 112 are formed of the relatively softest material. The goal is to have the plunger shaft 112 rub against the plunger guide 140 in the event of misalignment, rather than rubbing against the plunger bore 105. Wearing of the plunger guide 140 is preferable to wearing of any of the other components. As a result, the centering syringe plunger guide 140 will reduce unwanted wear to more critical (and harder to replace) components. This is desirable, as wear on the syringe barrel 104, on the plunger head 112, and/or on the plunger shaft 114 may result in greater damage over time than wear on the centering syringe plunger guide 140. Further, the centering syringe plunger guide 140 may be replaceable, and more easily replaceable than the syringe barrel 104 or the plunger shaft 114.

The centering syringe plunger guide 140 may be formed of a plastic material in some embodiments. The centering syringe plunger guide 140 may be formed of a bearing-grade plastic material in some embodiments. However, other materials are contemplated and are within the scope of the description and claims.

In some embodiments, the tolerance between the plunger shaft 114 and the guide plunger bore 145 may be relatively small. As a result, the centering syringe plunger guide 140 may further act like a seal. Alternatively, sufficient clearance may remain to allow air (or other fluids) to be drawn into and expelled from the plunger bore 105. In some embodiments, the centering syringe plunger guide 140 may include a vent hole.

FIG. 3 shows the self-centering syringe assembly 100 partially assembled, with the coupling sleeve 120 positioned on the barrel precision diameter 108 of the syringe barrel 104. The coupling sleeve 120 may be removably or permanently affixed to the syringe barrel 104 in this position, as was previously discussed. It can be seen from this figure that the barrel precision diameter 108 is larger than the axial dimension of the annular coupling sleeve 120. As a consequence, the coupling sleeve 120 does not occupy the full barrel precision diameter 108. A portion of the barrel precision diameter 108 is still exposed and receives the precision guide bore 144 of the centering syringe plunger guide 140.

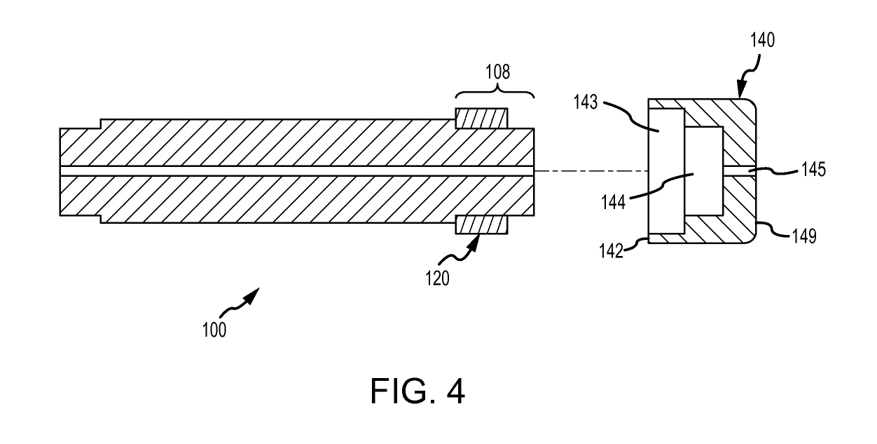

FIG. 4 shows a full section view AA of the self-centering syringe assembly 100 when partially assembled. As in FIG. 3, only the coupling sleeve 120 is positioned on the syringe barrel 104 in this example. This figure illustrates that the coupling bore 143 is configured to fit over and engage the coupling sleeve 120. This figure illustrates that the precision guide bore 144 is configured to fit over and engage the barrel precision diameter 108 of the syringe barrel 104. This figure illustrates that the guide plunger bore 145 is configured to align with and be centered on the plunger bore 105 of the syringe barrel 104.

FIG. 5 shows a full section view AA of the self-centering syringe assembly 100 when completely assembled. Here, the centering syringe plunger guide 140 has been fitted over the coupling sleeve 120, with the coupling bore 143 engaging the coupling sleeve 120. The precision guide bore 144 has been fitted over the protruding portion of the barrel precision diameter 108, with the engaging of the precision guide bore 144 with the barrel precision diameter 108 causing a proper alignment of the centering syringe plunger guide 140 with respect to the syringe barrel 104. Further, the guide plunger bore 145 is forced into a predetermined alignment. The guide plunger bore 145 is forced to be substantially coaxial with the plunger bore 105 as a result of the fit. The guide plunger bore 145 is forced to be substantially centered with the plunger bore 105 as a result of the fit.

The detailed descriptions of the above embodiments are not exhaustive descriptions of all embodiments contemplated by the inventors to be within the scope of the invention. Indeed, persons skilled in the art will recognize that certain elements of the above-described embodiments may variously be combined or eliminated to create further embodiments, and such further embodiments fall within the scope and teachings of the invention. It will also be apparent to those of ordinary skill in the art that the above-described embodiments may be combined in whole or in part to create additional embodiments within the scope and teachings of the invention. Thus, although specific embodiments of, and examples for, the invention are described herein for illustrative purposes, various equivalent modifications are possible within the scope of the invention, as those skilled in the relevant art will recognize. Accordingly, the scope of the invention should be determined from the following claims.

We claim:

1. A centering syringe plunger guide (140), comprising:
a guide body (141) including a proximal end (142) and a distal end (149);
a coupling bore (143) extending partially into the guide body (141) from the proximal end (142);
a precision guide bore (144) extending partially into the guide body (141) from the coupling bore (143), with the precision guide bore (144) adapted to fit over a barrel precision diameter of a syringe barrel and hold the centering syringe plunger guide (140) in a predetermined substantially coaxial alignment with the syringe barrel; and
a guide plunger bore (145) extending completely from the precision guide bore (144) to the distal end (149) of the guide body (141), with the guide plunger bore (145) being adapted to receive a syringe plunger and is configured to substantially center the syringe plunger in a plunger bore of the syringe barrel.

2. The centering syringe plunger guide (140) of claim 1, wherein the centering syringe plunger guide (140) is formed of a material having predetermined wear characteristics.

3. The centering syringe plunger guide (140) of claim 1, wherein the centering syringe plunger guide (140) is formed of a plastic material.

4. The centering syringe plunger guide (140) of claim 1, wherein the centering syringe plunger guide (140) is formed of a bearing-grade plastic material.

5. The centering syringe plunger guide (140) of claim 1, with the guide body (141) further including a guide textured region (154) on an external circumferential surface.

6. The centering syringe plunger guide (140) of claim 1, wherein the precision guide bore (144) holds the centering syringe plunger guide (140) in a predetermined substantially coaxial alignment with a plunger bore (105) of the syringe barrel (104).

7. The centering syringe plunger guide (140) of claim 1, wherein the precision guide bore (144) holds the centering syringe plunger guide (140) in a predetermined substantially coaxial alignment with a plunger bore (105) of the syringe barrel (104) and wherein the guide plunger bore (145) is smaller than the plunger bore (105).

8. The centering syringe plunger guide (140) of claim 1, with the centering syringe plunger guide (140) including a corresponding guide coupling feature (146) in the coupling bore (143).

9. The centering syringe plunger guide (140) of claim 1, wherein the precision guide bore (144) of the centering syringe plunger guide (140) is adapted to fit over the barrel precision diameter of the syringe barrel with a predetermined clearance.

10. The centering syringe plunger guide (140) of claim 1, wherein the coupling bore (143), the precision guide bore (144), and the guide plunger bore (145) are substantially coaxial.

11. The centering syringe plunger guide (140) of claim 1, wherein the coupling bore (143), the precision guide bore (144), and the guide plunger bore (145) are substantially coaxial and further are substantially coaxial with a guide body axis BB.

12. A self-centering syringe assembly (100) including a centering syringe plunger guide (140), with the self-centering syringe assembly (100) comprising:
a syringe barrel (104) including a plunger bore (105) and a barrel precision diameter (108) formed on one end of the syringe barrel (104);
a coupling sleeve (120) configured to fit over and be affixed to the barrel precision diameter (108) of the syringe barrel (104); and
a centering syringe plunger guide (140) configured to be affixed to the coupling sleeve (120), with the centering syringe plunger guide (140) comprising:
a guide body (141) including a proximal end (142) and a distal end (149);
a coupling bore (143) extending partially into the guide body (141) from the proximal end (142);
a precision guide bore (144) extending partially into the guide body (141) from the coupling bore (143), with the precision guide bore (144) being configured to fit over a barrel precision diameter (108) of a syringe barrel (104) and hold the centering syringe plunger guide (140) in a predetermined substantially coaxial alignment with the syringe barrel (104); and
a guide plunger bore (145) extending completely from the precision guide bore (144) to the distal end (149) of the guide body (141), with the guide plunger bore (145) being configured to receive a syringe plunger (114) and configured to substantially center the syringe plunger (114) in the plunger bore (105) of the syringe barrel (104).

13. The self-centering syringe assembly (100) of claim 12, wherein the centering syringe plunger guide (140) is formed of a material having predetermined wear characteristics.

14. The self-centering syringe assembly (100) of claim 12, wherein the centering syringe plunger guide (140) is formed of a plastic material.

15. The self-centering syringe assembly (100) of claim 12, wherein the centering syringe plunger guide (140) is formed of a bearing-grade plastic material.

16. The self-centering syringe assembly (100) of claim 12, with the guide body (141) further including a guide textured region (154) on an external circumferential surface.

17. The self-centering syringe assembly (100) of claim 12, wherein the precision guide bore (144) holds the centering syringe plunger guide (140) in a predetermined substantially coaxial alignment with a plunger bore (105) of the syringe barrel (104).

18. The self-centering syringe assembly (100) of claim 12, wherein the precision guide bore (144) holds the centering syringe plunger guide (140) in a predetermined substantially coaxial alignment with a plunger bore (105) of the syringe barrel (104) and wherein the guide plunger bore (145) is smaller than the plunger bore (105).

19. The self-centering syringe assembly (100) of claim 12, with the coupling sleeve (120) including a sleeve coupling feature (126) and with the centering syringe plunger guide (140) including a corresponding guide coupling feature (146) in the coupling bore (143).

20. The self-centering syringe assembly (100) of claim 12, wherein the precision guide bore (144) of the centering syringe plunger guide (140) is configured to fit over the barrel precision diameter (108) of the syringe barrel (104) with a predetermined clearance.

21. The self-centering syringe assembly (100) of claim 12, wherein the coupling bore (143), the precision guide bore (144), and the guide plunger bore (145) are substantially coaxial.

22. The self-centering syringe assembly (100) of claim 12, wherein the coupling bore (143), the precision guide bore (144), and the guide plunger bore (145) are substantially coaxial and further are substantially coaxial with a guide body axis BB.
